SEQUENCE LISTING (6 sequences)

SEQ ID NO: 1 - protein, 99 aa, Homo sapiens
  URG7
MAAPAEPCAGQGVWNQTEPEPAATSLLSLCFLRTAGVWVPPMYLWVLGPIYLLFIHHHGRGYLRMSPLFKAKMVAAIPGSLEPGNVRGRQGTGWNLVKS

SEQ ID NO: 2 - protein, 1503 aa, Homo sapiens
  MRP6
MAAPAEPCAGQGVWNQTEPEPAATSLLSLCFLRTAGVWVPPMYLWVLGPIYLLFIHHHGRGYLWMSPLFKAKMVLGFALIVLCTSSVAVALWKIQQGTPEAPEFLIHPTVWLTTMSFAVFLIHTERKKGVQSSGVLFGYWLLCFVLPATNAAQQASGAGFQSDPVRHLSTYLCLSLVVAQFVLSCLADQPPFFPEDPQQSNPCPETGAAFPSKATFWWVSGLVWRGYRRPLRPKDLWSLGRENSSEELVSRLEKEWMRNRSAARRHNKAIAFKRKGGSGMKAPETEPFLRQEGSQWRPLLKAIWQVFHSTFLLGTLSLIISDVFRFTVPKLLSLFLEFIGDPKPPAWKGYLLAVLMFLSACLQTLFEQQNMYRLKVLQMRLRSAITGLVYRKVLALSSGSRKASAVGDVVNLVSVDVQRLTESVLYLNGLWLPLVWIVVCFVYLWQLLGPSALTAIAVFLSLLPLNFFISKKRNHHQEEQMRQKDSRARLTSSILRNSKTIKFHGWEGAFLDRVLGIRGQELGALRTSGLLFSVSLVSFQVSTFLVALVVFAVHTLVAENAMNAEKAFVTLTVLNILNKAQAFLPFSIHSLVQARVSFDRLVTFLCLEEVDPGVVDSSSSGSAAGKDCITIHSATFAWSQESPPCLHRINLTVPQGCLLAVVGPVGAGKSSLLSALLGELSKVEGFVSIEGAVAYVPQEAWVQNTSVVENVCFGQELDPPWLERVLEACALQPDVDSFPEGIHTSIGEQGMNLSGGQKQRLSLARAVYRKAAVYLLDDPLAALDAHVGQHVFNQVIGPGGLLQGTTRILVTHALHILPQADWIIVLANGAIAEMGSYQELLQRKGALVCLLDQARQPGDRGEGETEPGTSTKDPRGTSAGRRPELRRERSIKSVPEKDRTTSEAQTEVPLDDPDRAGWPAGKDSIQYGRVKATVHLAYLRAVGTPLCLYALFLFLCQQVASFCRGYWLSLWADDPAVGGQQTQAALRGGIFGLLGCLQAIGLFASMAAVLLGGARASRLLFQRLLWDVVRSPISFFERTPIGHLLNRFSKETDTVDVDIPDKLRSLLMYAFGLLEVSLVVAVATPLATVAILPLFLLYAGFQSLYVVSSCQLRRLESASYSSVCSHMAETFQGSTVVRAFRTQAPFVAQNNARVDESQRISFPRLVADRWLAANVELLGNGLVFAAATCAVLSKAHLSAGLVGFSVSAALQVTQTLQWVVRNWTDLENSIVSVERMQDYAWTPKEAPWRLPTCAAQPPWPQGGQIEFRDFGLRYRPELPLAVQGVSFKIHAGEKVGIVGRTGAGKSSLASGLLRLQEAAEGGIWIDGVPIAHVGLHTLRSRISIIPQDPILFPGSLRMNLDLLQEHSDEAIWAALETVQLKALVASLPGQLQYKCADRGEDLSVGQKQLLCLARALLRKTQILILDEATAAVDPGTELQMQAMLGSWFAQCTVLLIAHRLRSVMDCARVLVMDKGQVAESGSPAQLLAQKGLFYRLAQESGLV

SEQ ID NO: 3 - protein, 25 aa, Homo sapiens
  Peptide75-99 URG7
AAIPGSLEPGNVRGRQGTGWNLVKS

SEQ ID NO: 4 - protein, 111 aa, Homo sapiens
  Polipeptide His-URG7
MRGSHHHHHHGSMAAPAEPCAGQGVWNQTEPEPAATSLLSLCFLRTAGVWVPPMYLWVLGPIYLLFIHHHGRGYLRMSPLFKAKMVAAIPGSLEPGNVRGRQGTGWNLVKS

SEQ ID NO: 5 - DNA, 28 bp, Homo sapiens
  Oligonucleotide forward primer 5′, 3′
tcgcggatccatggccgcgcctgctgag

SEQ ID NO: 6 - DNA, 32 bp, Homo sapiens
  Oligonucleotide reverse primer 5′, 3′
cgaaagctttcaagacttcaccaggttccagc

(19) United States
(12) Patent Application Publication
(10) Pub. No.: US 2017/0138949 A1
(43) Pub. Date: May 18, 2017

(54) METHOD FOR DIAGNOSING THE RISK OF PRENEOPLASTIC AND NEOPLASTIC LIVER DISEASE IN SUBJECTS AFFECTED BY HEPATITIS

(71) Applicant: UNIVERSITA' DEGLI STUDI DELLA BASILICATA, Potenza (IT)

(72) Inventors: Angela OSTUNI, Potenza (IT); Faustino BISACCIA, Vaglio di Basilicata (IT)

(21) Appl. No.: 15/321,759

(22) PCT Filed: Jun. 23, 2015
(86) PCT No.: PCT/EP2015/064043

§ 371 Date: Dec. 23, 2016

(30) Foreign Application Priority Data
Jun. 25, 2014 (IT) PZ2014A000005

(51) Int. Cl. G01N 33/574; G01N 33/68

(52) U.S. Cl. CPC G01N 33/57438; G01N 33/6854

(57) ABSTRACT

The invention herein described relates to an early and specific diagnosis method based on the use of the peptide fragment 75-99 of the URG7 protein. This peptide is used as an antigen to determine the presence of anti-URG7 antibodies in the sera of carrier subjects of the hepatitis virus, of hepatitis B in particular.

### FIELD OF THE INVENTION

The invention relates to a method for diagnosing the risk of preneoplastic and neoplastic liver diseases in patients affected by hepatitis virus infection based on the use of the peptide fragment 75-99 of the URG7 protein.

### BACKGROUND OF THE INVENTION

It is known that the hepatitis B virus contributes to determining the onset of serious pathological consequences, such as chronic diseases of the liver, cirrhosis and hepatocellular carcinoma, one or more proteins encoded by this virus playing a significant role in hepatocarcinogenesis. In particular, hepatitis and cirrhosis are among the most common risk factors of hepatocellular carcinoma (HCC), which is one of the most frequent cancers at global level and with limited diagnostic options. In fact, presently, the methods most used for diagnosing hepatocellular carcinoma are based on instrumental methods for ultrasound imaging and liver biopsies that detect visible cancers. However, both with the instrumental investigations and with biopsies, it is difficult to distinguish between cirrhotic nodules and a differentiated HCC or dysplastic HCC nodules having a low degree of differentiation. To overcome the limitations of these diagnostic methods, a method based on the dosage of alpha-fetoprotein in the blood has been developed, which finds support in the fact that the diagnosis of hepatocellular carcinoma was associated with a high concentration of this protein in the blood [McMahon B J, et al. Hepatology, 2000, 32, 842-846; Toyoda H. et al. Clin Gastroenterol Hepatol., 2006, 4, 1170-1176; Thompson Coon J et al. Health Technol Assess., 2007, 11, 1-206; Volk M L et al. Cancer Biomark., 2007, 3, 79-87]. However, a method of diagnosis based on the determination of this protein is strongly limited by its low specificity [Sheman M. et al. J Hepatol., 2001, 34, 603-5; Oka H. et al. Hepatology, 1994, 19, 61-66].

Since the efficacy of the treatment of hepatocellular carcinoma largely depends on an early diagnosis to the onset of the pathological condition, there is a great need to have a method capable of diagnosing any progression toward a hepatocellular carcinoma in the initial stage when it is not yet detectable using instrumental methods for images.

It has now been ascertained that the hepatitis B virus x antigen (HBxAg) is involved in the pathogenetic progression of chronic hepatitis into hepatocellular carcinoma. This antigen is capable of altering the gene expression profile of the host cell [Sui Hie-Won L. Hann, et al. Cancer Res., 2004, 64, 7329-7335]. Of the over-expressed proteins, a particularly important role appears to be played by the URG7 protein, encoded by the URG7 gene (up-regulated gene 7), which appears to play an active role in blocking the apoptotic processes of the infected cells induced by TNFα, interacting with the beta catenin and directly inhibiting caspases 3 and 8 [Pan J. et al. Journal of General Virology, 2007, 88, 3275-3285]. The determination of anti-URG7 antibodies, in addition to anti-Sui1, in the serum of patients affected by hepatitis B long before the diagnosis of HCC, could have important prognostic significance since the appearance thereof appears to be associated with the formation of dysplastic nodules rather than the risk of developing hepatocellular carcinoma (HCC) generally associated with the appearance of anti-URG11, anti-URG4 and anti-S15 antibodies [Hie-Won L. Hann et al. Cancer Res., 2004, 64, 7329-7335].

The URG7 protein consists of 99 amino acids and the gene that encodes it is a 2271-nucleobase gene [Lian Z. et al. Hepatology, 2001, 34, 146-157]. This gene was subsequently identified as the pseudogene 2 of the ABCC6 gene, a gene that encodes a transport protein of 1503 amino acids, MRP6, which mutations are associated with the onset of pseudoxanthoma elasticum, a connective tissue disorder [Li Q. et al. Matrix Biol., 2014, 33C, 23-28]. Studies carried out on the ABCC6 gene have demonstrated the existence of truncated forms of ABCC6 arising from alternative splicing processes [Armentano, M. F. et al. Research Letters in Biochemistry, 2008, doi:10.1155/2008/912478]. It has also been demonstrated that the ABCC6 gene is over-expressed in certain human cancer cell lines [Kool M. et al. Cancer Res., 1999, 59, 175-182] and, recently, in some cancers, such as non-small cell lung cancer and ovarian cancer after chemotherapy treatment. Moreover, its expression is associated with reduced apoptotic activity induced by anticancer drugs [Ikeda R. et al. Int. J. Oncol., 2011, 38, 513-9; Luo H. et al. Cancer Cell lnt., 2010, 10, 16].

Since the amino acid sequence of the URG7 protein is identical in the first 74 residues with the MRP6 protein, it must be established with certainty if the anti-URG7 antibodies found in patients affected by hepatitis B [Hie-Won L. Hann et al. 2004, ref. cit.] are due to the over-expression of URG7 or of MRP6. At present, the detection of anti-URG7 antibodies in patients infected with HBV is performed using antigens as the N-terminal sequencesl2-25 and 56-70 of URG7 [Hie-Won L. Hann et al., 2004, ref. cit; US Patent No. 2007/0048808] that coincide with the N-terminal 12-25 and 56-70 sequences of the MRP6 protein.

Although immunochemical methods and kits are widely used to detect early markers of disease their use is often limited by poor specificity. The search for ever-more specific tests is therefore a basic necessity for a correct diagnosis and the present invention allows identification of the presence of anti-URG7 antibodies as a specific preneoplastic marker.

### SUMMARY OF THE INVENTION

One purpose of the present invention is, therefore, the development of a diagnostic method based on the use of an antigenic peptide that belongs to the URG7 protein and is non superimposable to the sequence of the MRP6 protein, to detect specific anti-URG7 antibodies in the serum of patients affected by chronic hepatitis infection, and by HBV in particular.

In one aspect, the present invention concerns a method for diagnosing the risk of preneoplastic and neoplastic liver diseases in subjects affected by hepatitis virus infection, by hepatitis B in particular, based on detecting specific antibodies against the peptide fragments 75-99 of the URG7 protein consisting of the peptide with the aaipgs lepgnvrgrq gtgwnlvks sequence identified as SEQ ID NO:3, in a biological sample, preferably serum, of these subjects.

Therefore, in a first aspect the object of the invention is a method for diagnosing the risk of preneoplastic and neoplastic liver diseases in subjects affected by hepatitis virus infection comprising at least the step of:
determining in a biological sample the presence of anti-URG7 antibodies by detection thereof with the peptide of SEQ ID NO:3, wherein the presence of antibodies in the biological sample is indicative of a low risk of progression from chronic hepatitis to preneoplastic and neoplastic liver diseases in subjects affected by viral hepatitis.

In another aspect, the object of the present invention is an immunochemical kit for detecting specific antibodies against the URG7 protein in a biological sample, preferably serum, of patients affected by hepatitis virus infection, in particular HVB, comprising at least: one container with the peptide of SEQ ID NO:3, consisting of the peptide fragment 75-99 of the URG7 protein, one container with an anti-human IgG secondary antibody conjugated with a label for detecting the signal and an instructions for use.

The method and the kit according to the invention allow an early diagnosis to be made, and with non-invasive methods, when the neoplasia is not yet detectable using instrumental images.

### BRIEF DESCRIPTION OF THE DRAWINGS

FIG. 1. The figure shows the absorbance values at 450 nm detected by means of indirect ELISA testing of the serum of healthy subjects, in the presence of the antigen of SEQ ID NO:3 (indicated with □ PEP) and of the antigen of SEQ ID NO:4 (indicated with ▪ POLYPEP).

FIG. 2. The figure shows the absorbance values at 450 nm detected by means of indirect ELISA testing of the serum of patients affected by hepatitis B infection in the presence of the antigen of SEQ ID NO:3 (indicated with □ PEP) and of the antigen of SEQ ID NO:4 (indicated with ▪ POLYPEP).

### DETAILED DESCRIPTION OF THE INVENTION

Definitions

Here, “URG7 protein” means human isoform 2, member of the MRP subfamily of the superfamily of ABC transport proteins (ATP-binding cassette) encoded by the URG7 gene [NCBI accession number NC_000016]. The URG7 protein is the protein identified in the database of the National Center for Biotechnology Information with the access number NP_001072996; the sequence is reported in the sequences listing as SEQ ID NO:1.

“Protein MRP6” means the human protein member of the MRP subfamily of the ABC (ATP-binding cassette) transporter protein superfamily involved in multidrug resistance phenomena. The protein is encoded by the ABCC6 [Homo sapiens Gene ID 368] gene. In the National Center for Biotechnology Information database it is identified with the GenBank accession number AAC79696; the sequence is reported in the sequences listing as SEQ ID NO:2.

Description

The present invention concerns a method for evaluating the presence of antibodies against the 75-99 sequence of the URG7 protein in the serum of patients affected by hepatitis virus infection, in particular by hepatitis B, at risk of developing both a less serious pathological form such as dysplastic liver nodules and more serious forms of liver disease such as cirrhosis and hepatocellular carcinoma.

Essentially, the method according to the invention is based on an immunochemical dosage by means of indirect ELISA (Enzyme-Linked Immunosorbent Assay) assay of the anti-URG7 antibodies in serum samples from patients affected by hepatitis. The immunochemical assay can be performed according to the methods known to a person skilled in the art and, in particular, an immunochemical dosage based on the detection of a radiochemical, fluorescent or enzymatic signal by means of spectrophotometric, fluorimetric or chemiluminescent measurements.

This is a versatile and widely used immunological analysis method that allows to detect the presence and dosage of an antigen (direct ELISA) or of a specific antibody against an antigen (indirect ELISA).

Therefore, in one embodiment of the invention, the method for diagnosing the risk of preneoplastic and neoplastic diseases in subjects affected by hepatitis virus infection, and preferably by hepatitis B, comprises at least the step of determining the presence of anti-URG7 antibodies by detection thereof with the peptide of SEQ ID NO:3 in a serum sample of these subjects.

The risk assessment of a progression of the disease from chronic hepatitis to precancerous or neoplastic diseases in patients affected by chronic viral hepatitis, by hepatitis B in particular, is carried out by comparing the results obtained on the samples of subjects affected by this disease with samples (sera) obtained from healthy subjects. Determinations of the anti-URG7 antibodies on healthy subjects can be carried out autonomously to obtain standard reference values or during the diagnostic test.

In a preferred embodiment the antigen is bound to a support selected from a polystyrene plate, nitrocellulose filter, but chips, microarrays, functionalized resins can be used.

For the purposes of the method according to the invention, it is necessary to immobilize on a plate at least 700 ng, and preferably 1 pg of the peptide 75-99, and at least 50 ng and preferably 200 ng of the His-URG7 polypeptide (SEQ ID NO:4) used as control.

The signal is detected by means of an anti-human IgG secondary antibody conjugated with a marker enzyme, such as for example horseradish peroxidase, alkaline phosphatase, B-galactosidase, glucose oxidase, urease, or derivatized with a fluorophore, such as fluorescein and tetramethylrhodamine for example.

As regards the detection systems, in order to increase sensitivity, reduce the consumption of reagents and sample and reduce the analysis time, biosensors based on advanced technologies of the optical type, such as surface plasmon resonance (SPR), or electrochemical devices such as microelectrodes, can also be used.

Therefore, in a preferred embodiment, the method for diagnosing the risk of preneoplastic and neoplastic diseases in subjects affected by hepatitis virus infection, preferably of type B, provides that the determination of the presence of anti-URG7 antibodies, by means of the detection thereof with the peptide of SEQ ID NO:3, in a biological sample of these subjects comprises the steps of:
binding the antigen consisting of the peptide of SEQ ID NO:3 to a solid support;
adding the biological sample, preferably serum, of the subject affected by hepatitis virus infection and reacting with the immobilized antigen;
adding an anti-human IgG secondary antibody conjugated with a label;
detecting the signal resulting from the reaction of antigen/specific anti-URG7 antibodies (primary antibodies)/ labelled anti-human IgG secondary antibody;
comparing the value of the signal detected in the biological sample of the subject affected by hepatitis virus infection against the average value of the signal previously detected in samples, preferably serum, of healthy control subjects.

Optionally, the method can comprise the further step of adding a biological sample (serum) of one or more healthy control subjects.

A value of the signal, substantially absorbance at 450nm, greater than that of the average of the healthy control samples (sera) more than twice the standard deviation, is considered a positive value, indicative of the presence of specific antibodies in assayed serum.

Optionally, as a positive control, the His-URG7 polypeptide (SEQ ID NO:4) can also be immobilized on a similar support. The supports with this polypeptide are then treated like the supports treated with the peptide of SEQ ID NO:3 and the procedure to be followed is entirely similar to what has previously been described for this antigen.

The method for diagnosing the risk of preneoplastic and neoplastic liver diseases in subjects affected by hepatitis virus infection according to the invention can therefore comprise the further steps of:
binding the antigen consisting of the polypeptide of SEQ ID NO:4 to a solid support;
adding the biological sample and reacting with the immobilized antigen;
adding an anti-human IgG secondary antibody conjugated with a label; detecting the signal resulting from the reaction of antigen/specific anti-URG7 antibodies (primary antibodies)/ labelled anti-human IgG secondary antibody;
comparing the value of the signal detected in the biological sample of the subject affected by infection at the signal value detected in a biological sample of a healthy control subject.

The method for diagnosing the risk of preneoplastic and neoplastic diseases in subjects affected by hepatitis virus infection B based on the detection of anti-URG7 antibodies can be achieved with a kit comprising at least: a container containing the antigen consisting of the peptide of SEQ ID NO:3, optionally in combination with a second container containing an anti-human IgG secondary antibody conjugated with a label, and a an information leaflet of the procedure and with an indication of the standard values of these anti-URG7 antibodies in healthy control subjects. Optionally, the kit may further comprise a container with the polypeptide His-URG7 of SEQ ID NO:4 as control. Preferably, both the peptide of SEQ ID NO:3 and the polypeptide of SEQ ID NO:4 are bound to a suitable support for carrying out the analysis.

### Experimental Part

### EXAMPLE 1

### Solid Phase Synthesis of Peptide 75-99 of the URG7 Protein (SEQ ID NO:3)

Before proceeding to the synthesis of the peptide 75-99 specific for the URG7 protein, prediction studies for the presence of epitopes were carried out using different software available on the network. The evaluation of the immunogenic potential of an amino acid sequence is carried out by taking various parameters, such as flexibility, accessibility, degree of hydrophilicity and homology with known epitopes, into consideration. The most reliable information was obtained using the ABCPRED software, which exploits a database (Bcipep) of amino acid sequences recognised as epitopes and capable of triggering an immune response in a host organism and of reacting with the products of this immune response. From the epitope prediction study it has emerged that the non-homologous region of 25 amino acids between the URG7 polypeptide and the MRP6 protein could possess a potential immunogenicity, and could therefore be used for the production of a specific antibody specific for the URG7 polypeptide.

The peptide corresponding to the region 75-99 of the URG7 protein was synthesized using the solid-phase synthesis method with Fmoc strategy on an automatic Pioneer PerSeptive Biosystems synthesizer. Novabiochem amino acids were used with the amino groups protected by the Fmoc (9-flurenyl-methoxycarbonyl) protecting group removed in a basic environment (20% piperidine in dimethylformamide, DMF). Protection of the amino acid amine group is essential during activation of the carboxylic group so that the latter reacts exclusively with the free amino group on the last amino acid residue of the growing peptide chain. An amino acid:functional group of the resin molar ratio equal to 4:1 was used. The resin employed is Fmoc-PAL-PEG-PS (Applied Biosystem), consisting of a copolymer of polystyrene and polyethylene glycol. Binding of the first amino acid to the resin is facilitated by a linker of the PAL-type (Fmoc-aminomethyl-2,5-dimethoxy-phenoxy valeric acid).

Assembly of the amino acid chain consists of a series of cyclic deprotection reactions of the amino protector group of the residue bound to the growth and condensation peptide between the amino group and the activated carboxyl function of a new residue, with formation of a peptide bond. Amino acids, beyond the amino terminus and the carboxylic acid terminus, can contain further reactive groups in the side chain: a protection strategy of the side chains with groups that prove stable will therefore be necessary. The reagent employed for activation of the carboxylic function was PyBOP (benzotriazole-1-yl-oxytripyrrolidinophosphonium hexafluorophosphate) (0.5 M in DMF) in the presence of DIPEA (N,N-diisopropylethylamine) (1M in DMF).

On completion of the synthesis, the peptide was detached from the resin and the protector groups of the amino acid side chains were simultaneously removed using crystalline phenol 0.75 g, ethanedithiol 0.25 ml, thioanisole 0.25 ml, deionized water 0.5 ml, trifluoroacetic acid 10 ml (TFA).

After 3 hours of reacting at room temperature, the suspension was filtered on a semipermeable membrane, adding 2 ml of TFA and 4 ml of dichloromethane (DCM). The filtrate containing the peptide was concentrated in a rotavapor at a temperature of about 30° C., and the peptide was precipitated with ethyl ether, isolated by centrifugation solubilised in H20/TFA 0.1% and CH3CN/TFA 0.1% in a ratio of 1:1 and, lastly, lyophilized. The peptide was then purified by reversed-phase high performance liquid chromatography (RP-HPLC) on a C18 column (Jupiter 5 μm 300 Å, 250×10.00 mm, Phenomenex) and with a gradient of the mobile phase, consisting of Acetonitrile+0.1% TFA, water+TFA ranging from 5% to 60% acetonitrile in 30 minutes, maintaining the concentration at 60% for 10 minutes and returning to 5% acetonitrile in 5 minutes. The flow rate is equal to 3 ml/min. The fraction relating to the peptide was collected, freeze-dried and analysed by electrospray mass spectrometry.

### EXAMPLE 2

### Preparation of the His-URG7 Polypeptide (SEQ ID NO:4) by Recombinant DNA Technology

The His-URG7 polypeptide was amplified by PCR from cDNA of hepatoma HepG2 cells using as forward primer 5′-TCGCGGATCCATGGCCGCGCCTGCTGAG-3′ (SEQ ID NO:5) and as reverse primer 5′-CGAAAGCTTTCAAGACTTCACCAGGTTCCAGC-3′ (SEQ ID NO:6). The gene was then cloned in the expression vector pQE30, transformed into Rosetta pLYS-S E.coli strain (Novagen), and expressed as His-tagged polypeptide to the N-terminal (His-URG7). Purification was carried out by means of RP-HPLC [Ostuni A. et al. Prot. Pept. Lett., 2014, 21 (5), 413-8].

### EXAMPLE 3

### Production of the Specific Polyclonal Antibody for the Peptide 75-99 of SEQ ID NO:3

The peptide 75-99 URG7 with an N-terminal cysteine residue (Cys-URG7) conjugated with the protein carrier, KLH (Keyhole Limpet Hemocyanin), and mixed in a 1:1 ratio with alum (aluminium hydroxide) as adjuvant was used to immunize white rabbits (New Zealand) according to the standard procedure implemented by the company PRIMM srl (Milan). Each rabbit was subcutaneously injected with a dose of immunogen equal to 250 pg. The following challenges with 200 pg of conjugate were carried out: a first follow-up at a distance of three weeks and three other follow-ups at a distance of one week from the another.

The serum (S) was provided as a such and also purified by means of affinity chromatography (A).

### EXAMPLE 4

### Evaluation of the Antigen Immobilization Methodology and of the Immunochemical Reactivity Thereof by Means of the ELISA Test

In order to determine the optimal conditions to be used in the assay to detect any specific anti-URG7 antibodies in the serum of patients, preliminary indirect ELISA experiments were carried out, fixing onto a plate both the peptide 75-99 (SEQ ID NO:3) and the His-URG7 polypeptide (SEQ ID NO:4) at different concentrations and using different dilutions of both the immune rabbit serum (S) and the purified anti-URG7 IgG antibodies (A). Different experimental conditions were considered in order to optimize the binding of the antigen and solubilisation of the peptide 75-99 and the His-URG7 polypeptide in order to keep them in the immunochemically active form.

The optimal experimental procedure is described below:
1. add of 100 μL of glutaraldehyde at 5% onto a multiwell plate (Nunc Brand Products-Polysorp) and incubate for 1 hour at room temperature;
2. gently empty the wells and perform 5 washes with 1×PBS (200 μl/well);
3. in accordance with the plate diagram add 100 μl of antigen: use a 10 μg/ml solution of the peptide 75-99 in PBS 1× and use a 2 μg/ml solution of His-URG7 in 70% TFE;
4. incubate the plate at 4° C. overnight;
5. gently empty the wells and perform 3 washes with PBS 1× (200 μL per well);
6. add 200 μl of a solution of 5% powdered skimmed milk in PBS 1× to all the wells in which the assay is to be performed and incubate for 2 hours at room temperature;
7. gently empty the wells and perform 3 washes with PBS 1× (200 μl per well);
8. in accordance with the plate diagram add 50 μl of a solution of serum and purified antibody, suitably diluted in PBS 1% powdered skimmed milk and incubate the plate for 1 hour at room temperature;
9. gently empty the wells and perform 8 washes with PBST 1× (Tween 20 at 0.05% (200 μL per well);
10.in accordance with the plate diagram add 50 μl of a solution of secondary antibody (anti-rabbit IgG-HRP Sigma) diluted1:5,000 in PBS 1% powdered skimmed milk;
11. incubate the plate for 1 hour at room temperature;
12.gently empty the wells and perform 8 washes with PBST 1× (Tween 20 at 0.05% (200 μL per well);
13.add 50 μl of a solution of OPD (o-phenylenediamine dihydrochloride) in citrate phosphate buffer pH 9.6 (0.8 mg of OPD in 1 ml of citrate phosphate buffer) to each well;
14. leave the plate in the dark for 30 minutes and perform a spectrophotometer reader (λ=450 nm).

The amount of antigen (1 μg for the peptide 75-99 and 200 ng for His-URG7) was maintained fixed and the dilutions 1:100, 1:1000, 1:3000, 1:7000, 1:20000 and 1:50000 for the serum (S) and the concentrations 2 μg/ml, 1 μg/ml, 0.5 μg/ml, 0.1 ρg/ml, 0.05 μg/ml and 0.01 μg/ml for the purified IgG antibodies (A), were tested.

The absorbance values at 450 nm in the indirect ELISA assay using the immune rabbit serum (S) and the purified anti-URG7 antibodies (A) are indicated in TABLE I (A and B, respectively):

TABLE I A

| | | | | | | | Nega- |
|---|---|---|---|---|---|---|---|
| ANTI- | DILUTION OF RABBIT IMMUNE SERUM | | | | | | tive |
| GENS | 1/100 | 1/1000 | 1/3000 | 1/7000 | 1/20000 | 1/50000 | control |
| peptide | 1.368 | 1.110 | 0.663 | 0.287 | 0.103 | 0.057 | 0.221 |
| 75-99 | | | | | | | |
| His- | 1.389 | 1.369 | 0.987 | 0.505 | 0.251 | 0.103 | 0.246 |
| URG7 | | | | | | | |

TABLE I B

| | PURIFIED ANTIBODY CONCENTRATIONS | | | | | | Nega- |
|---|---|---|---|---|---|---|---|
| ANTI- | 2 | 1 | 0.5 | 0.1 | 0.05 | 0.01 | tive |
| GENS | μg/μL | μg/μL | μg/μL | μg/μL | μg/μL | μg/μL | control |
| peptide | 1.153 | 0.842 | 0.448 | 0.129 | 0.060 | 0.063 | 0.221 |
| 75-99 | | | | | | | |
| His- | 1.376 | 1.203 | 0.681 | 0.270 | 0.143 | 0.002 | 0.246 |
| URG7 | | | | | | | |

From analysis of the data obtained it has been possible to establish that the minimum amount of immune rabbit serum and purified antibody used, if the peptide 75-99 (1 μg) or His-URG7 (200 ng) is immobilized on the plate, is as reported in TABLE II:

TABLE II

| | peptide 75-99 | His-URG7 |
|---|---|---|
| IMMUNE SERUM | 1/7000 | 1/20000 |
| Purified antibodies | 0.5 μg/μL | 0.1 μg/μL |

While keeping constant the dilution of the serum (1:7000) and of the purified anti-URG7 antibodies (0.5 μg/ml), various amounts (from 1 μg to 50 ng) of both the peptide 75-99 (Table III A) and of the His-URG7 polypeptide (TABLE III B) were immobilized on a plate in order to determine the minimum optimal amount of antigen for detecting the formation of the specific antigen and antibody complex. The absorbance values recorded in the ELISA assays carried out are as follow:

TABLE III A

| | peptide 75-99 | | | | | | Nega- |
|---|---|---|---|---|---|---|---|
| PRIMARY | 1 | 700 | 400 | 200 | 100 | 50 | tive |
| ANTIBODY | μg | ng | ng | ng | ng | ng | control: |
| IMMUNE | 0.212 | 0.106 | 0.088 | 0.022 | 0.012 | 0.004 | 0.210 |
| SERUM | | | | | | | |
| Purified | 0.444 | 0.178 | 0.077 | 0.045 | 0.011 | 0.003 | 0.246 |
| antibodies | | | | | | | |

TABLE III B

| | His-URG7 | | | | | | Nega- |
|---|---|---|---|---|---|---|---|
| PRIMARY | 1 | 700 | 400 | 200 | 100 | 50 | tive |
| ANTIBODY | μg | ng | ng | ng | ng | ng | control |
| IMMUNE | 0.641 | 0.614 | 0.578 | 0.554 | 0.460 | 0.355 | 0.210 |
| SERUM | | | | | | | |
| Purified | 1.165 | 1.100 | 1.033 | 0.965 | 0.799 | 0.677 | 0.246 |
| antibodies | | | | | | | |

Analysis of the data obtained has made it possible to determine that plate immobilization of 1 pg of peptide 75-99 and at least 50 ng of the His-URG7 polypeptide is necessary.

### EXAMPLE 5

### Evaluation of the Presence of Specific anti-URG7 Antibodies in the Sera of Healthy Patients Affected by Hepatitis B by Means of Indirect ELISA

After optimizing the binding conditions of the two antigens (plate activated with glutaraldehyde and a minimum amount of the antigen to be depositing), stability over time was evaluated (at 4° C. and for at least 3 months). In collaboration with IRCSS C.R.O.B. of Rionero, 18 sera from healthy subjects and 30 sera from patients affected by HBV infection (supplied by operational Extended Care Unit of San Francesco Hospital in Venosa) were assayed.

The ELISA protocol that was followed is the same as the one reported in example 4, but with the following variants:
point 3: use a solution of the peptide 75-99 10 pg/ml in 1× PBS (1 pg per well) and a solution of His-URG7 in 70% TFE 2 pg/ml (200 ng/well);
point 8: add 100 μl of the patient's serum diluted 1:50 in PBS 1% powdered milk;
point 10: use, as secondary antibody, rabbit anti-human IgG-HRP (Santa Cruz, sc-2769).

All the tests were carried out in triplicate and the appropriate negative controls (absorbance evaluation in wells with the patient's serum only and antibody II but without an antigen) were also carried out.

Results

So as not to influence evaluation of the results, a “blind” study was carried out, i.e. the investigator was not aware of the clinical status of the patient whose serum was being assayed. The result of the immunochemical dosage and the clinical data was then associated.

The sera of 12 healthy subjects (8 females with average age of 39±9; 4 males with average age of 43±4) and 30 sera of patients affected by HBV infection (3 females 3 with average age of 72±16; 27 males with average age of 51±11) were examined. All the patients examined proved HbsAg positive, anti-HDV negative, with normal alpha-fetoprotein and only 14 under conventional pharmacological regimen (with interferon and/or nucleotide/nucleosides analogues).

In the case of active carriers (19 in the study) the values of HBV-DNA at the onset ranged from a minimum of 299 IU/ml to a maximum of approximately 160,000000 IU/ml. In most cases, after pharmacological treatment, HBV-DNA converted from positive to negative or at most remained at the values of 396 IU/ml (7,600,000 at onset). The sera evaluated were all obtained from patients HBV-DNA negative patients.

In 6 HBV-DNA positive patients, a diagnosis of cirrhosis was made, while only 1 turned out to be affected by hepatocellular carcinoma due to HBV infection and 1 patient was affected by hepatocellular carcinoma from HCV (with cirrhosis and died before commencing a therapeutic programme).

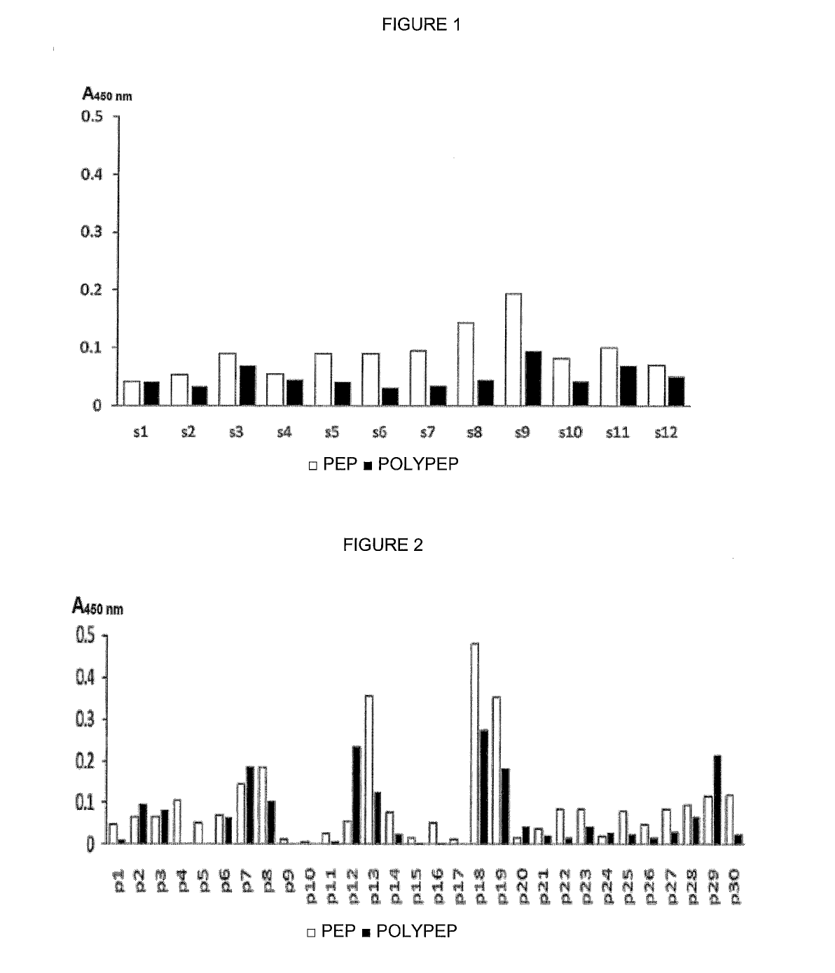

Under optimised experimental conditions, an absorbance value greater than the average absorbance value of the healthy control serum (FIG. 1) plus twice the standard deviation was considered a positive value, indicative of the presence of specific antibodies in the serum assayed. From the results obtained, it is observed that of the 5 sera positive for the His-URG7 polypeptide (patients 7, 12, 18, 19, 29), only 2 were also positive for the peptide 75-99 (patients 18 and 19), while the serum of patient 13 is only positive for the peptide 75-99 (FIG. 2).

For two of these patients (patients 13 and 19), neither cirrhosis or HCC was diagnosed, despite the high levels of HBV-DNA at the onset. In patient 18, with a high level of infection at the onset, there is cirrhosis but no progression towards HCC in a year of treatment with Baraclude®. Only one patient with a high level of infection at diagnosis (patient 8), manifested cirrhosis that rapidly evolved into HCC and the anti-URG7 antibody levels for the His-URG7 polypeptide were found to be no different from those of the healthy subjects and at the limit of positivity for the peptide 75-99.

1. A method for diagnosis of the risk of preneoplastic and neoplastic liver diseases in subjects affected by viral hepatitis comprising at least the step of:
determining in a biological sample the presence of anti-URG7 antibodies by detection thereof with the peptide of SEQ ID NO:3 consisting of the peptide fragment 75-99 of the URG7 protein, wherein the presence of the anti-URG7 antibodies in the biological sample is indicative of a low risk of progression from chronic hepatitis to preneoplastic and neoplastic liver diseases in said subjects.

2. The method for diagnosis of the risk of preneoplastic and neoplastic liver diseases in subjects affected by viral hepatitis according to claim 1, wherein the presence of the anti-URG7 antibodies in the assayed sample is evaluated with reference to a signal value resulting from the detection thereof higher than the average signal values obtained on biological samples of healthy subjects plus twice the standard deviation.

3. The method for diagnosis of the risk of preneoplastic and neoplastic liver diseases in subjects affected by viral hepatitis according to claim 1, wherein the step of determining the presence of the anti-URG7 antibodies is carried out by means of an ELISA assay.

4. The method for diagnosis of the risk of preneoplastic and neoplastic liver diseases in subjects affected by viral hepatitis according to claim 1, wherein the biological sample is serum.

5. the method for diagnosis of the risk of preneoplastic and neoplastic liver diseases in subjects affected by viral hepatitis according to claim 1, comprising the steps of:
binding the antigen consisting of the peptide od SEQ ID NO:3 to a solid support;
adding a biological sample of a subject affected by viral hepatitis and reacting with the immobilized antigen;
adding an anti-human IgG secondary antibody conjugated with a label;
detecting he signal resulting from the reaction of antigen/specific anti-URG7 antibodies (primary antibodies)/labeled anti-human IgG secondary antibody;
comparing the value of the signal detected in the biological sample of the subject affected by viral hepatitis with the average signal values obtained on biological samples of healthy subjects.

6. The method for diagnosis of the risk of preneoplastic and neoplastic liver diseases in subjects affected by viral hepatitis according to claim 1, wherein the polypeptide of DEQ ID NO:4 consisting of the His-URG7 polypeptide is used as a positive control.

7. The method for diagnosis of the risk of preneoplastic and neoplastic liver diseases in subjects affected by viral hepatitis according to claim 6, comprising the further steps of:
binding the antigen consisting of the peptide of SEQ ID NO:4 to a solid support;
adding a biological sample of a subject affected by viral hepatitis and reacting with the immobilized antigen;
adding an anti-human IgG secondary antibody conjugated with a label;
detecting the signal resulting from the reaction of antigen/specific anti-URG7 antibodies (primary antibodies)/labeled anti-human IgG secondary antibody;
comparing the value of the signal detected in the biological sample of the subject affected by viral hepatitis with the average signal values obtained on biological samples of healthy subjects.

8. The method for diagnosis of the risk of preneoplastic and neoplastic liver diseases in subjects affected by viral hepatitis according to claim 1, wherein the hepatitis is due to the hepatitis B virus.

9. The method for diagnosis of the risk of preneoplastic and neoplastic liver diseases in subjects affected by viral hepatitis according to claim 1, wherein the preneoplastic and neoplastic liver diseases are cirrhosis, hepatocellular carcinoma.

10. A kit for detection of anti URG7 antibodies in a biological sample, preferably serum, of a subject affected by viral hepatitis, comprising at least:
a container with the peptide of SEQ ID NO:3, consisting of the peptide fragment 75-99 of the URG7 protein; and
an information leaflet.

11. The kit for detection of anti URG7 antibodies according to claim 10, comprising a further container with an anti-human IgG secondary antibody conjugated with a label for detecting the signal.

12. The kit for detection of antibodies anti URG7 according to claim 10, comprising a further container with the polypeptide of SEQ ID NO:4 consisting of the His-URG7 polypeptide.

13. The kit for detection of antibodies anti URG7 protein according to claim 10, wherein the peptide of SEQ ID NO:3 and the polypeptide of SEQ ID NO:4 are bound to a support.
